(19) United States
(12) Patent Application Publication
(10) Pub. No.: US 2016/0199299 A1
(43) Pub. Date: Jul. 14, 2016

(54) Cannabis Infused Chewing Composition

(71) Applicant: Mark B. Uren, The Colony, TX (US)

(72) Inventor: Mark B. Uren, The Colony, TX (US)

(21) Appl. No.: 14/707,507

(22) Filed: May 8, 2015

Related U.S. Application Data
(60) Provisional application No. 62/101,885, filed on Jan. 9, 2015.

(51) Int. Cl. A61K 9/00; A61K 31/05; A61K 47/46; A24B 13/00; A61K 47/10; A61K 47/22; A61K 36/185; A61K 31/352; A61K 47/02

(52) U.S. Cl. CPC A61K 9/0056; A61K 31/352; A61K 31/05; A61K 47/46; A61K 47/02; A61K 47/10; A61K 47/22; A61K 36/185; A24B 13/00

(57) ABSTRACT

A cannabis infused chewing composition imparting extracts from cannabis plants to a user for medicinal and recreational purposes. The cannabis infused chewing composition includes a quantity of cannabis plant matter, a quantity of cannabis extract, a quantity of bonding composition, a quantity of moisturizer, and a quantity of flavor enhancer. The quantity of cannabis plant matter, the quantity of cannabis extract, the quantity of moisturizer, and the quantity of flavor enhancer are homogenously amalgamated into a chewable mixture by the quantity of bonding composition. A quantity of shredded tobacco can be included to impart nicotine and other desired tobacco extracts to the user. The shredded tobacco is infused with the quantity of cannabis extracts in a similar manner to the quantity of cannabis plant matter and heterogeneously combined with the chewable mixture.

The current application claims a priority to the U.S. Provisional Patent application Ser. No. 62/101,885 filed on Jan. 9, 2015.

### FIELD OF THE INVENTION

The present invention relates generally to a cannabis infused smokeless tobacco composition. More specifically, the present invention relates to a composition for cannabis infused chew products that can be utilized in a medicinal or recreational manner.

### BACKGROUND OF THE INVENTION

Chewing tobacco, also known as chew, dip, snuff, and other various names, is consumed by users that wish to imbibe tobacco without smoking tobacco products. Chewing tobacco is consumed by users by chewing the product then placing it in between the cheek and gum, upper lip and teeth, or lower lip and teeth. Unlike cigarettes, the chemicals extracted from chewing tobacco are absorbed through oral tissue allowing the user to feel the effects of the chemicals at a much quicker rate.

Currently, there are not any similar chewable products for cannabis or any active ingredients. The consumption of compounds found in cannabis provide therapeutic effect to those with eating disorders, sleep disorders, anxiety issues, neurodegenerative disorders, multiple sclerosis, and other ailments. In particular, cannabidol (CBD) and tetrahydrocannabinol (THC) are compounds in cannabis which have many medicinal benefits. Unlike THC, CBD is not considered to be a psychotropic compound, which affects a person's mental state.

It is an object of the present invention to provide a chewable cannabis substance for recreational or medicinal use. Cannabis plant matter, including buds (also known as nubs), leaves, roots, and stems, is infused with additional cannabis extracts and oils to increase the potency of the compounds found within a cannabis plant. The cannabis plant matter is shredded, pressed, or ground into a chewable composition. Tobacco is an addition to some embodiments to provide the user with nicotine while providing the medicinal and recreational effects of the compounds found in cannabis plants.

### BRIEF DESCRIPTION OF THE DRAWINGS

FIG. 1 is a block diagram illustrating the compounds of the present invention.

FIG. 2 is a block diagram illustrating the compounds of an alternate embodiment for the present invention.

FIG. 3 is a block diagram illustrating the composition of cannabis plant matter.

FIG. 4 is a block diagram illustrating the compounds of the quantity of cannabis extracts.

FIG. 5 is a block diagram illustrating the compounds of the quantity of moisturizer.

FIG. 6 is a block diagram illustrating the compounds of the quantity of flavor enhancer.

FIG. 7 is a block diagram illustrating the compounds of the quantity of additives.

### DETAIL DESCRIPTIONS OF THE INVENTION

All illustrations of the drawings are for the purpose of describing selected versions of the present invention and are not intended to limit the scope of the present invention.

The present invention is for a cannabis infused chewing composition which imparts chemical compounds to the user through the oral tissue as the present invention is chewed. Chemical compounds from cannabis provide the user with therapeutic effects to those with eating disorders, sleep disorders, anxiety issues, neurodegenerative disorders, multiple sclerosis, and other ailments. The present invention is processed into fresh cut, long cut, pressed, individual portioned pouches, or other similar forms appropriate for a chewable cannabis applicator.

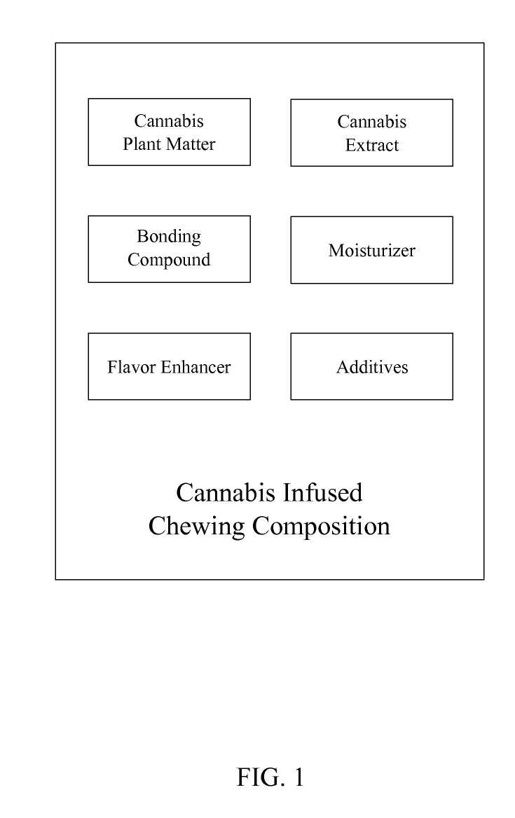

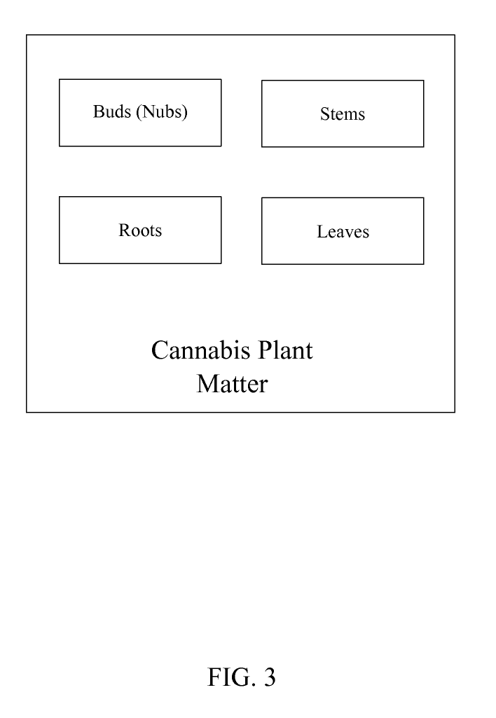

In accordance to FIG. 1, the present invention comprises a quantity of cannabis plant matter, a quantity of cannabis extract, a quantity of bonding composition, a quantity of moisturizer, and a quantity of flavor enhancer. The quantity of cannabis plant matter provides a solid base for the quantity of cannabis extract, the quantity of moisturizer, and the quantity of flavor enhancer to be sorbed onto through use of the quantity of bonding composition, as well as providing an initial portion of cannabis extracts. The quantity of cannabis plant matter is selected from a group consisting of cannabis buds (also known as nubs), leaves, roots, stems, or combinations thereof, as shown in FIG. 3. The quantity of cannabis extract provides additional amounts of select extracts for medicinal and recreational uses. The quantity of moisturizer keeps the quantity of cannabis plant matter from drying out. The quantity of flavor enhancer imparts sweet and salty tastes to the user allowing the present invention to be more palatable. The quantity of cannabis plant matter, the quantity of cannabis extract, the quantity of moisturizer and the quantity of flavor enhancer are homogeneously amalgamated into a chewable mixture by the bonding composition.

Through chewing the chewable mixture and contact with saliva, the active chemical compounds from the quantity of cannabis plant matter and the quantity of cannabis extract are separated from the chewable mixture. The active chemical compounds are then absorbed through the oral tissue of the user.

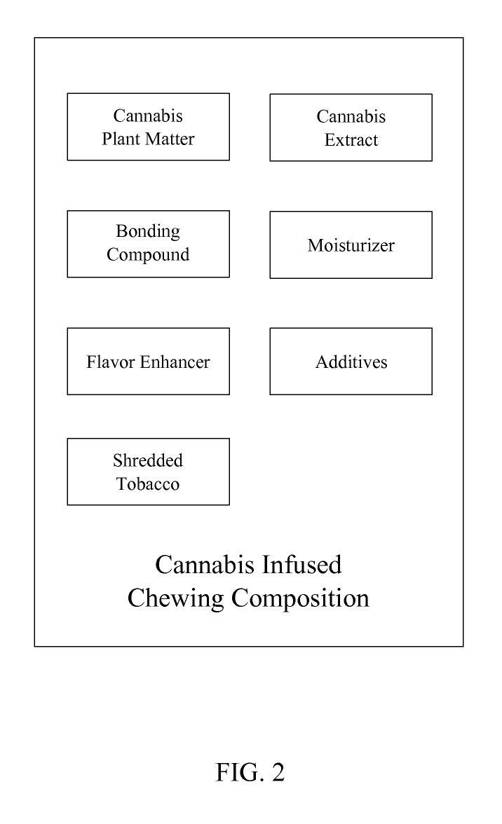

In accordance to an alternate embodiment, shown in FIG. 2, the present invention further comprises a quantity of shredded tobacco. The quantity of shredded tobacco provides the user with tobacco flavorings and nicotine, as well as an additional base on which the quantity of cannabis extract, the quantity of moisturizer, the quantity of flavor enhancer are sorbed. The quantity of cannabis extract, the quantity of moisturizer, and the quantity of flavor enhancer are homogeneously integrated into the quantity of shredded tobacco by the bonding composition. The quantity of shredded tobacco is then heterogeneously integrated into the chewable mixture.

Similar to the preferred embodiment, the active chemical compounds from the quantity of cannabis plant matter, the quantity of shredded tobacco, and the quantity of cannabis extract are separated from the chewable mixture, through chewing the chewable mixture and contact with saliva. The active chemical compounds are then absorbed through the oral tissue of the user.

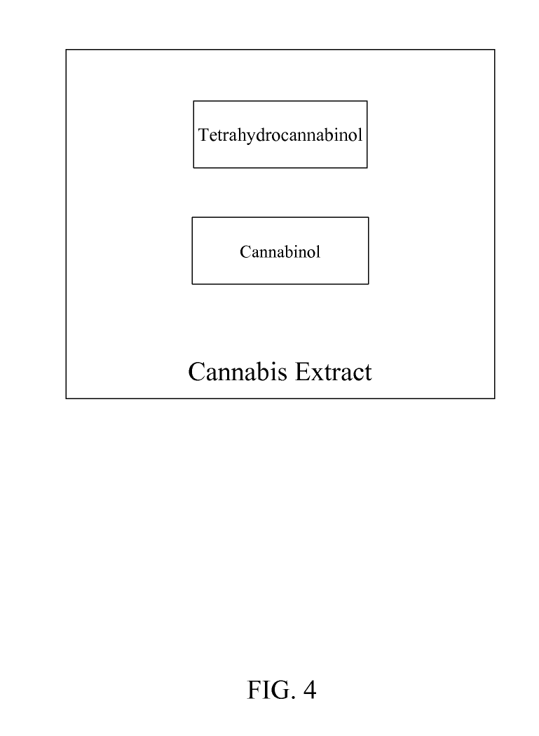

In accordance to FIG. 4, the quantity of cannabis extract is active extracts selected from a group consisting of tetrahydrocannabinol (THC), cannabinol (CBD), and combinations thereof. THC is a psychotropic compound found in cannabis plants selected for recreational embodiments, inducing euphoria, and medicinal embodiments, treating multiple sclerosis (MS) symptoms and neurodegenerative disorders, of the present invention. CBD has wider scope of medicinal applications over THC and does not provide the user with a euphoric feeling. Depending on the intent of the application, the ratio of THC to CBD within the quantity of cannabis extract is selected between 0:1 and 1:0 THC to CBD, more recreational and more medicinal respectively.

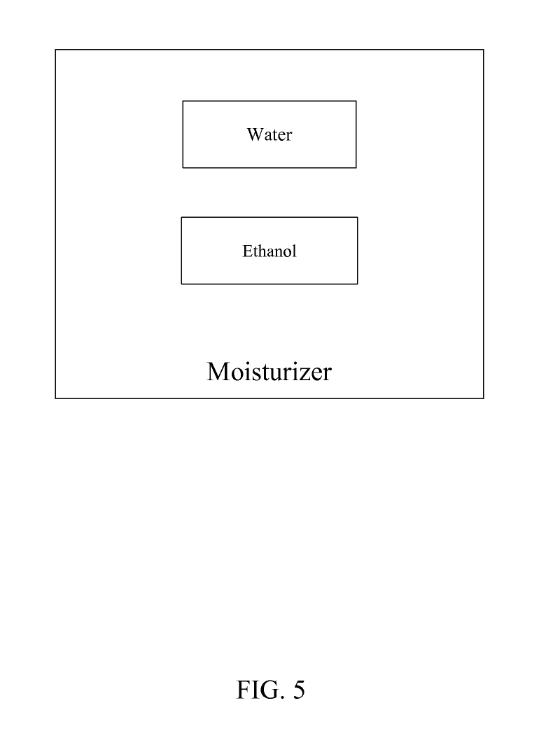

As previously mentioned, the quantity of moisturizer prevents the present invention from drying out easily. In accordance to FIG. 5, the quantity of moisturizer is selected from a group consisting of water, ethanol, and combinations thereof. Water and alcohol are selected because they are easily absorbed into the organic matter of the quantity of cannabis plant matter and the quantity of shredded tobacco. Keeping the chewable mixture moist allows for the present invention to be easier to chew as well as providing an initial medium for the active chemical compounds from the quantity of cannabis plant matter and the quantity of cannabis extract, as well as the quantity of shredded tobacco in the alternate embodiment, before contacting saliva.

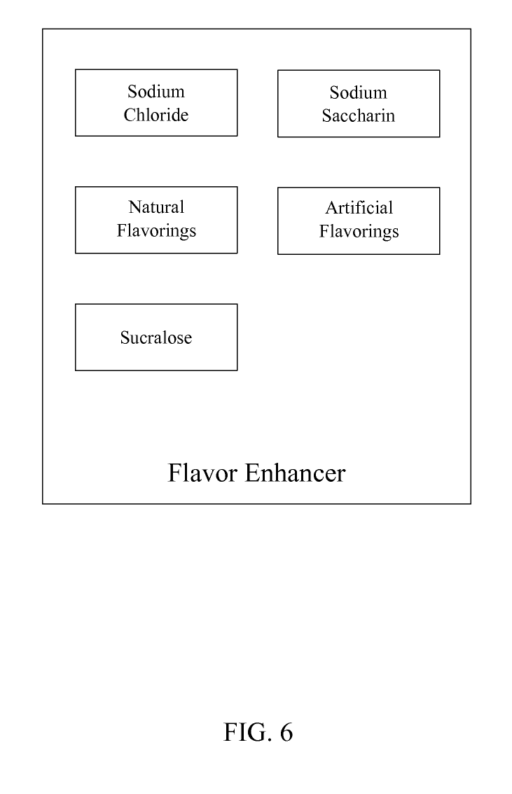

The quantity of flavor enhancer counteracts some of the tart and unpalatable tastes from the quantity of cannabis plant matter, as well as the quantity of shredded tobacco in an alternate embodiment. In accordance to FIG. 6, the quantity of flavor enhancer is selected from a group consisting of sodium chloride, sodium saccharin, natural flavorings, artificial flavorings, sucralose, and combinations thereof. The sodium chloride, commonly known as table salt, imparts a salty taste to the chewable mixture. Sodium chloride is also a preservative to extend the shelf life of the present invention. Sodium saccharin and sucralose sweeten the chewable mixture, while natural flavorings and artificial flavorings alter the taste to give the impression of foods or other substances. Natural flavoring and artificial flavorings include, but are not limited to, fruit flavors, nut flavors, bean flavors, and other appropriate flavor additives.

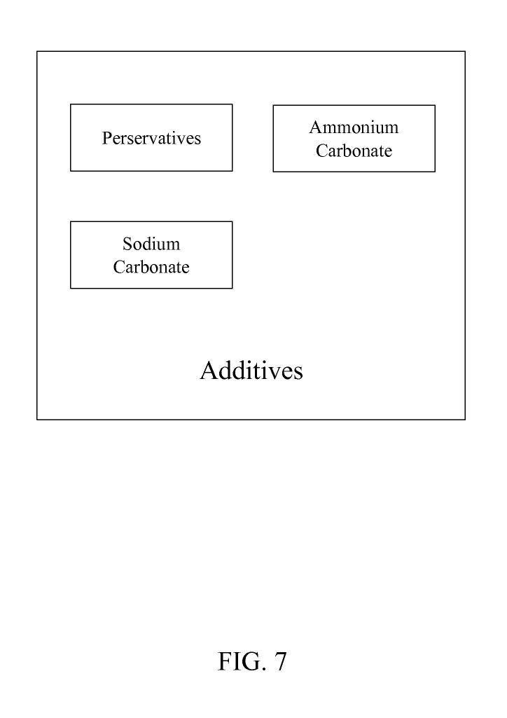

Further in accordance to the preferred embodiment, the present invention further comprises a quantity of additives, as detailed in FIG. 7. The quantity of additives is inactive compounds which exert properties for extending the shelf life and adjusting the acidity of the chewable mixture. The quantity of additives is selected from a group consisting of preservatives, ammonium carbonate, sodium carbonate, and combinations thereof. Preservatives extend the shelf life of the present invention. Ammonium carbonate and sodium carbonate increase the pH of the chewable mixture, alleviating the bitter tastes from the quantity of cannabis plant matter and the quantity of shredded tobacco when chewed.

In order to manufacture the present invention, the quantity of cannabis plant matter is ground into a sufficiently small particle size forming ground cannabis. The ground cannabis is then soaked in the quantity of cannabis extract, the quantity of moisturizer, and the quantity of bonding composition forming an infused cannabis mixture. The infused cannabis mixture is dried to remove excess mixture. The quantity of flavor enhancer and the quantity of additives are mixed into the dried infused cannabis mixture forming the chewable mixture.

In embodiments comprising the quantity of shredded tobacco, the quantity of shredded tobacco is similarly ground into smaller particles forming ground tobacco. The ground cannabis and the ground tobacco are then soaked in the quantity of cannabis extracts, the quantity of moisturizer, and the quantity of bonding composition forming an infused cannabis tobacco mixture. The infused cannabis tobacco mixture is dried to remove excess mixture. The quantity of flavor enhancer and the quantity of additives are mixed into the dried infused cannabis tobacco mixture forming the chewable mixture.

Although the invention has been explained in relation to its preferred embodiment, it is to be understood that many other possible modifications and variations can be made without departing from the spirit and scope of the invention as hereinafter claimed.

What is claimed is:

1. A cannabis infused chewing composition comprises:
a quantity of cannabis plant matter;
a quantity of cannabis extract;
a quantity of bonding composition;
a quantity of moisturizer;
a quantity of flavor enhancer; and
the quantity of cannabis plant matter, the quantity of cannabis extract, the quantity of moisturizer, and the quantity of flavor enhancer being homogeneously amalgamated into a chewable mixture by the quantity of bonding composition.

2. The cannabis infused chewing composition as claimed in claim 1 comprises:
a quantity of shredded tobacco;
the quantity of cannabis extract, the quantity of moisturizer, and the quantity of favor enhancers being homogeneously integrated into the quantity of shredded tobacco by the quantity of bonding composition; and
the quantity of shredded tobacco being heterogeneously integrated into the chewable mixture.

3. The cannabis infused chewing composition as claimed in claim 1 comprises:
the quantity of cannabis plant matter being selected from a group consisting of cannabis buds, leaves, roots, stems, or combinations thereof.

4. The cannabis infused chewing composition as claimed in claim 1 comprises:
the quantity of cannabis extract being selected from a group consisting of tetrahydrocannabinol (THC), cannabinol (CBD), and combinations thereof.

5. The cannabis infused chewing composition as claimed in claim 1 comprises:
the quantity of moisturizer being selected from a group consisting of water, ethanol, and combinations thereof.

6. The cannabis infused chewing composition as claimed in claim 1 comprises:
the quantity of flavor enhancer being selected from a group consisting of sodium chloride, sodium saccharin, natural flavorings, artificial flavorings, sucralose and combinations thereof.

7. The cannabis infused chewing composition as claimed in claim 1 comprises:
a quantity of additives; and
the quantity of additives being homogeneously integrated into the chewable mixture.

8. The cannabis infused chewing composition as claimed in claim 7 comprises:
the quantity of additives being selected from a group consisting of preservatives, ammonium carbonate, sodium carbonate, and combinations thereof.

9. A cannabis infused chewing composition comprises:
a quantity of cannabis plant matter;
a quantity of cannabis extract;
a quantity of bonding composition;
a quantity of moisturizer;
a quantity of flavor enhancer;
a quantity of additives; and
the quantity of cannabis plant matter, the quantity of cannabis extract, the quantity of moisturizer, the quantity of flavor enhancer, and the quantity of additives being homogeneously amalgamated into a chewable mixture by the quantity of bonding composition.

10. The cannabis infused chewing composition as claimed in claim 9 comprises:
a quantity of shredded tobacco;
the quantity of cannabis extract, the quantity of moisturizer, and the quantity of favor enhancers being homogeneously integrated into the quantity of shredded tobacco by the quantity of bonding composition; and
the quantity of shredded tobacco being heterogeneously integrated into the chewable mixture.

11. The cannabis infused chewing composition as claimed in claim 9 comprises:
the quantity of cannabis plant matter being selected from a group consisting of cannabis buds, leaves, roots, stems, or combinations thereof.

12. The cannabis infused chewing composition as claimed in claim 9 comprises:
the quantity of cannabis extract being selected from a group consisting of tetrahydrocannabinol (THC), cannabinol (CBD), and combinations thereof.

13. The cannabis infused chewing composition as claimed in claim 9 comprises:
the quantity of moisturizer being selected from a group consisting of water, ethanol, and combinations thereof.

14. The cannabis infused chewing composition as claimed in claim 9 comprises:
the quantity of flavor enhancer being selected from a group consisting of sodium chloride, sodium saccharin, natural flavorings, artificial flavorings, sucralose and combinations thereof.

15. The cannabis infused chewing composition as claimed in claim 9 comprises:
the quantity of additives being selected from a group consisting of preservatives, ammonium carbonate, sodium carbonate, and combinations thereof.
